(19) United States
(12) Patent Application Publication
(10) Pub. No.: US 2021/0098086 A1
(43) Pub. Date: Apr. 1, 2021

(54) SYSTEM AND METHOD FOR CONVERSATIONAL DATA COLLECTION

(71) Applicant: Universal Research Solutions, LLC, Columbia, MO (US)

(72) Inventor: Ali Adel Hussam, Columbia, MO (US)

(21) Appl. No.: 16/995,620

(22) Filed: Aug. 17, 2020

Related U.S. Application Data
(60) Provisional application No. 62/887,580, filed on Aug. 15, 2019.

(51) Int. Cl. G16H 10/20; G16H 20/30; G16H 10/60; G16H 50/50; G06Q 10/06; G16H 50/20; G06F 3/0482

(52) U.S. Cl. CPC G16H 10/20; G16H 20/30; G16H 10/60; G06F 3/0482; G06Q 10/067; G16H 50/20; G16H 50/50

(57) ABSTRACT

Methods, systems, and computer programs, for conversational data collection using digital questionnaire forms. In one aspect, a method comprises: providing a medical question of one or more medical questions in a medical questionnaire form; receiving first selection data describing a selection of the medical question; providing one or more response options corresponding to the medical question; obtaining first positional data describing a position of a user input mechanism; providing a visualization of a first medical condition; obtaining second positional data describing a different position of the user input mechanism; providing an update to the graphical user interface; receiving second selection data of the one or more response options; and storing, in one or more fields of a data structure used to represent answers to the one or more medical questions in the medical questionnaire form in a memory device, data that corresponds to the second selection data.

### CROSS-REFERENCE TO RELATED APPLICATIONS

This application claims the benefit of U.S. Provisional Patent Application No. 62/887,580, filed on Aug. 15, 2019. The entire contents of which is hereby incorporated by reference in their entireties.

### FIELD

This specification is generally related to systems and methods for conversational data collection using digital questionnaire forms.

### BACKGROUND

Users are asked to provide their information as responses to questionnaires in varied circumstances. For example, medical patients can be asked to provide their medical history or other health information by responding to medical questionnaire forms. Conventional questionnaire forms are presented as documents with questions and corresponding answer options in textual format, which can fail to engage the target audience, leading to challenges in collecting user data.

### SUMMARY

According to one innovative aspect, the subject matter of this specification is embodied in a method for conversational data collection. The method includes the actions of providing, for display on a graphical user interface of a user device, a medical question of one or more medical questions in a medical questionnaire form; receiving first selection data describing a selection of the medical question that was provided for display of the graphical user interface; in response to receiving the first selection data describing the selection of the medical question, providing, for display on the graphical user interface, one or more response options corresponding to the medical question; obtaining, at a first time, first positional data describing a position of a user input mechanism corresponding to a first particular option of the one or more response options; in response to the obtaining the first positional data at the first time, providing, for display on the graphical user interface, a visualization of a first medical condition representing the first particular option; obtaining, at a second time, second positional data describing a different position of the user input mechanism corresponding to a second particular option of the one or more response options; in response to obtaining the second positional data at the second time, providing an update to the graphical user interface, where the update causes the graphical user interface to display a visualization of a second medical condition representing the second particular option; receiving second selection data describing a selection of an option of the one or more response options; and in response to receiving the second selection data describing the selection of the option, storing, in one or more fields of a data structure used to represent answers to the one or more medical questions in the medical questionnaire form in a memory device, data that corresponds to the second selection data.

Other aspects include corresponding systems, apparatus, and computer programs to perform the actions of methods, encoded on computer storage devices.

Particular implementations may optionally include one or more of the following features. For example, in some implementations, the actions includes providing for display, on the graphical user interface, each of the one or more medical questions in a same order as an order in which the one or more medical questions are included in the medical questionnaire form.

In some implementations, the medical questionnaire form is a digital representation of a standard medical form, and the order of the one or more medical questions in the medical questionnaire form corresponds to an order of presentation of medical questions in the standard medical form.

In some implementations, the one or more response options correspond to different physical activities, the first particular option corresponding to a first physical activity and the second particular option corresponding to a different second physical activity and providing the visualization of the first medical condition includes providing, for display on the graphical user interface, a visualization of the first physical activity, and providing the visualization of the second medical condition includes providing, for display on the graphical user interface, a visualization of the second physical activity.

In some implementations, the one or more response options correspond to different intensity levels of a physical condition experienced by a user, the first particular option corresponding to a first intensity level and the second particular option corresponding to a different second intensity level, and providing the visualization of the first medical condition includes providing, for display on the graphical user interface, a visualization of the first intensity level, and providing the visualization of the second medical condition includes providing, for display on the graphical user interface, a visualization of the second intensity level.

In some implementations, providing, for display on the graphical user interface, at least one of: the medical question, the first medical condition, or the second medical condition, includes providing, for display on the graphical user interface, a multimedia animation.

In some implementations, the actions include providing, for display on the graphical user interface, the one or more medical questions and the visualizations in a narrative form as part of a storyline, and the actions further include obtaining information from a user interacting with the graphical user interface; in response to the information, selecting a particular storyline of one or more available storylines, the particular storyline corresponding to the user and representing a simulated version of a real-world environment; providing, for display on the graphical user interface, a first multimedia panel of the particular storyline, where the first multimedia panel includes (i) graphical content corresponding to a medical question of the one or more medical questions, and (ii) input options to enable interaction with the graphical content; receiving user input data indicating a selection one of the input options; determining a user action by processing the user input data; in response to determining the user action to be a first action: selecting a second multimedia panel of the particular storyline, and providing, for display on the graphical user interface, the second multimedia panel; and in response to determining the user action to be a different second action: selecting a different third multimedia panel of the particular storyline, and providing, for display on the graphical user interface, the third multimedia panel.

In some implementations, the actions further include determining whether at least one medical question of the one or more medical questions in the medical questionnaire form is unanswered; and based on determining that at least one medical question in the medical questionnaire form is unanswered, providing, for display on the graphical user interface, an unanswered medical question.

In some implementations, the actions further include determining whether at least one medical question of the one or more medical questions in the medical questionnaire form is unanswered; and based on determining that no medical question is unanswered: reviewing selection data corresponding to the one or more medical questions stored in the one or more fields of the data structure; computing a score using the selection data; and providing, for display on the graphical user interface, a summary of the medical questionnaire form, the summary including the computed score and a corresponding medical analysis.

The subject matter disclosed by this specification provides multiple advantages over conventional approaches. In some cases, providing digital representations of questionnaire forms using conversational prompts can cause greater user engagement, leading to more comprehensive, or more accurate, or both, collection of user data. Graphical visualizations of questions or answer options, or both, can help the target user better understand the rationale or focus of the questions, facilitating more accurate answers. For example, providing graphical visualizations depicting a medical condition covered by a question, with different animations to indicate different levels of intensity of the medical condition that patients normally experience, can help a patient contextualize her situation better, enabling her to provide a more precise answer. Providing graphical visualizations can also make the questionnaire forms entertaining and cause the user to be more attentive in answering the questions. Providing gamification for the questionnaire forms can further increase user engagement. For example, providing graphical visualizations that simulate real-world environments, with the user as a character in the simulated environment, can significantly increase user engagement, causing the user to provide answers more diligently or causing more users to answer questions, or both. Gamification can be particularly useful when the target audience is more difficult to engage. For example, using gamification for pediatric questionnaire forms can increase engagement from young patients, such as patients who are children. Accordingly, the systems and methods described herein provide for improving the quantity of user data collected in response to questionnaire forms, or improving the accuracy of the collected user data, or both. Analysis of the user data consequently can yield more accurate results for various uses, for example, leading to better treatment options for patients in medical settings.

### BRIEF DESCRIPTION OF THE DRAWINGS

FIG. 1 is a diagram of an example of a system for conversational data collection using a digital questionnaire form.

FIGS. 2A-2E illustrate examples of panels of a questionnaire form with graphical visualizations of questions and corresponding answer options that are provided for display on a graphical user interface shown on a user device.

FIG. 3 is a diagram of an example of a system for conversational data collection using a digital questionnaire form with gamification.

FIGS. 4A-4E illustrate examples of panels that are provided to display, on a graphical user interface shown on a user device, a digital questionnaire form in the form of a storyline with gamification.

FIG. 5 illustrates examples of panels that are provided to display, on a graphical user interface shown on a user device, a digital questionnaire form in the form of a storyline with gamification.

FIG. 6 illustrates an example of a process for conversational data collection using a digital questionnaire form with graphical visualizations.

FIG. 7 illustrates an example of a process for conversational data collection using a digital questionnaire form with gamification.

FIG. 8 is a diagram of computing devices that can be used to implement a system for conversational data collection using digital questionnaire forms.

Like reference numbers in the drawings indicate like elements.

### DETAILED DESCRIPTION

In some implementations, the present disclosure provides a model for conversational data collection, including collecting user data using digital questionnaire forms with graphical visualizations corresponding to question and answer options that are presented in a conversational form. In some implementations, the digital questionnaire forms are gamified. In such implementations, the question and answer options for a questionnaire form are presented within visualizations of a storyline representing a simulation of a real-world environment. In some implementations, characters are included in the visualizations to represent target users of the questionnaires.

The present disclosure provides significant technical advantages over conventional methods. In particular, implementations of the present disclosure described herein enables greater user engagement, which as a direct consequence, causes the system to obtain a more comprehensive and accurate user data. This is particularly useful in scenarios where accuracy of the user data is important, such as when collecting user data from medical patients using medical questionnaire forms. Accuracy and completeness of the collected patient data can help achieve better results of data analysis, enabling more precise tailoring of future treatment options for the affected patients, or in general to improve treatment options for the implicated medical conditions.

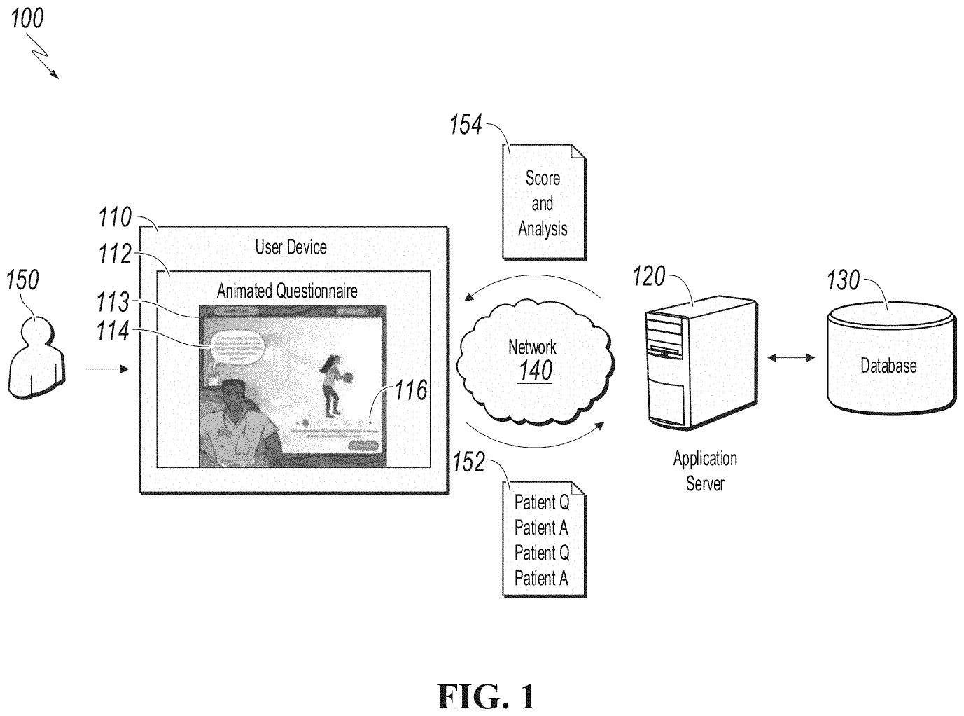

FIG. 1 is a diagram of an example of a system 100 for conversational data collection using a digital questionnaire form. The system 100 includes at least a user device 110, an application server 120 that is connected to the user device through a network 140, and a database 130 that is coupled to the application server 120.

The user device 110 is an electronic device with a display on which a graphical user interface (GUI) can be presented, and which further includes a network interface for connecting to the application server 120 over a network, such as the network 140. For example, the user device 110 can be a desktop computer, a laptop computer, a tablet computer, a smartphone, an electronic book reader, or a music player.

In some implementations, a GUI 112 is shown on the display of the user device 110. The GUI 112 provides graphical panels for a digital questionnaire form, which includes graphical visualizations for questions and answer options. For example, as shown, the GUI 112 includes a panel 113 of a medical questionnaire form, providing a graphical visualization 114 of a question being asked by a physician, and also providing a graphical visualization in the form of a scale where the scale shows a number of answer options 116 for the question. In other implementations, other forms of graphical interactive elements may be provided to the user 150 such as selectable items, bubbles of text, moveable items, or the like. A user such as user 150 interacting with the user device 110 can view the graphical visualizations in the panel 113 to interpret the question 114 and the answer options 116. The user 150 can select from one of the answer options 116 as the user's response to the question 114. Examples of digital questionnaire forms presented on the GUI 112 are described in greater detail below.

In the example of FIG. 1, the user 150 selects an option from among the answer options 116. Patient data 152 may include, depending on implementation, data corresponding to the particular answer option(s) selected by the user 150, data corresponding to the animated questionnaire shown on the GUI 112 of the user device 110, data corresponding to the user 150, or any combination thereof. In some implementations, other data can also be included within the patient data. For example, a device identifier corresponding to the user device 110 can be obtained and sent with the patient data 152 as a part of the patient data 152 or as a separate data packet. The patient data 152 can be sent from the user device 110 over the network 140 to the application server 120. The application server 120 is communicably connected to the database 130.

In the example of FIG. 1, the application server 120 receives patient data 152 related to the animated questionnaire shown on the GUI 112 of the user device 110 and the interaction of the user 150 with the animated questionnaire shown on the GUI 112 of the user device 110. The application server 120 can process the patient data 152 together with one or more stored data entries of the database 130 as discussed below. Based on the processing of the patient data 152, the application server 120 can generate a score and analysis report 154. The application server 120 can send the score and analysis report 154 over the network 140 to the user device 110. The user device 110 can receive the score and analysis report 154 and activate a corresponding display on the GUI 112 of the user device 110 to show one or more graphical elements associated with the score and analysis report 154 or the animated questionnaire.

In some implementations, the user device 110 can process the score and analysis report 154 and perform one or more additional operations. Processing the score and analysis report can include, for example, identifying and extracting one or more data items stored within the score and analysis report 154 and using the extracted one or more data items to generate and show a new panel to the user 150. In some implementations, the additional operations can include display of information included in the score and analysis report 154 in a panel. In other implementations, however, the user device 110 can generate and show a new panel similar to the panel 113 but including one or more different questions, answer options, visualizations, or a combination thereof, based on the content of the score and analysis report 154.

Although only one user device 110 is shown in FIG. 1, the system 100 can include a plurality of such user devices 110. In some implementations, the questionnaire forms are medical questionnaire forms for patients. For example, in some implementations, user 150 is a medical patient and one or more instances of user device 110 correspond to one or more of a computer at a physician's office that provides questionnaires that the physician's patients have completed, a third party server (or other computer) that aggregates medical questionnaires completed by patients of multiple different physicians, or client computers that belong to particular patients who have used respective computer devices to complete and transmit a questionnaire.

In some implementations, user device 110 can include one or more processors and one or more memory devices storing instructions. In some implementations, the one or more processors execute instructions stored in the one or more memory devices to run standalone application processes on the user device 110. The applications can correspond to operations to provide the GUI 112, including presenting panels with graphical visualizations for questions and answer options for questionnaire forms. In some of these implementations, the user device 110 analyzes the answers selected by the user 150, and dynamically determines, based on the selected answers, subsequent panels of questions to be presented on the GUI 112. Different answers selected by the user 150 can cause the user device 110 to present different panels on the GUI 112.

In some implementations, the one or more processors can execute instructions stored in the one or more memory devices to communicate data, application commands, or both, with the application server 120 over the network 140. For example, instructions can be executed by the one or more processors of the user device 110 to receive application commands from the application server 120, and process the application commands to provide the GUI 112. Presentation of the GUI 112 can include, for example, presenting panels with graphical visualizations for questions and answer options for questionnaire forms.

In some implementations, responses that are received from the user 150 as answers to questions shown on the GUI 112 are stored in the memory of the user device 110. In some implementations, an instance of the questionnaire form corresponding to the user 150, with answers selected by the user for the questions in the form, can be collected from the memory and sent, over the network 140, to the application server 120 as patient data 152.

In some implementations, responses that are received from the user 150 as answers to questions shown on the GUI 112 are forwarded to the application server 120 as the responses are received. In such implementations, each response, or one or more responses together, can be sent to the application server 120 as patient data 152. In some cases, the application server 120 can analyze the answers selected by the user 150, and dynamically determine, based on the selected answers, subsequent application commands to send to the user device 110 for presenting questions on the GUI 112. Different answers selected by the user 150 can cause the application server 120 to send application commands for presenting different questions.

The application server 120 includes one or more computing devices that are configured to receive documents, for example, patient data 152, from one or more remote computers. In some implementations, the documents include completed questionnaire forms for individual users with answer options selected by the users to a set of questions. In other implementations, the documents include iterative updates to questionnaire forms for individual users with answer options selected by the users to subsets of questions in each iteration.

As noted above, in some implementations, the documents include medical instruments such as medical questionnaire forms for patients. Each user device 110 can provide patient data such as one or more patient questionnaire forms to the application server 120 over the network 140. Such patient data can be stored and transmitted using data structures having fields that structure the patient data. For example, in some implementations, the patient data can be patient questionnaire forms stored as database records, tables, spreadsheets, or the like that have fields structuring question-answer pairs of the patient questionnaire form. By way of another example, defined protocol data units (PDUs) can be designed to organize the structure and transmission of the patient data across the network 140 from the user device 110 to the application server 120 or from the application server 120 to the user device 110. Patient data can be stored in the application server 120, database 130, or elsewhere using data structures in the same manner as that used by the user device 110. In some implementations, the network 140 represents one or more networks such as a LAN, a WAN, a cellular network, the Internet, or a combination thereof.

In some implementations, the patient data 152 shown in FIG. 1 can include medical questionnaires. The questionnaires can include one or more question-answer pairs. Each question-answer pair can include a question that was part of an administered questionnaire and a corresponding answer provided by the user 150. In some implementations, the application server 120 can include a data extraction unit that is configured to receive patient data 152 such as medical questionnaires that have been submitted, completed, or both, by patients. In implementations where the patient data 152 is a medical questionnaire, the data extraction unit can process the received medical questionnaire and store the received medical questionnaire in a patient data database, such as questionnaire database 130. The questionnaire database 130 can include any type of database such as a relational database, a hierarchical database, an unstructured database, or the like that allows for the storage and retrieval of the received patient questionnaires or other forms of patient data.

In some cases, the patient data 152 can include personal or account data related to the user 150. Personal data can include, for example, patient name, date of birth, or the like of the user 150. In some cases, an identifier of the user 150, such as a name or other alphanumeric type string, can be associated with one or more medical questionnaires answered by the user 150. The personal data of the user 150 can be stored in the database 130 along with one or more medical questionnaires answered by the user 150 or one or more medical questionnaires answered by other users.

In some implementations, patient data such as a medical questionnaire form, which includes question-answer pairs that have answers populated based on data provided by a user such as user 150 as an answer to a question of the question-answer pair, is stored as an entry in the database 130. In some implementations, answer options selected by the user 150 can be stored as data in one or more fields corresponding to the entry. The entry can be a database record.

In some implementations, storage of the received patient data 152 such as one or more medical questionnaires can include generating an index or an index entry that can be used to access one or more database records, each of which correspond to particular patient data 152 such as a received medical questionnaire form. In some cases, an index can be generated and configured to access one or more database records associated with a particular user or a particular group of one or more users.

Each index entry can include, for example, a user identifier, one or more keywords extracted from a received questionnaire form, and a form location that includes a reference to the storage location of the questionnaire identified by the index entry. In cases involving medical questionnaire forms, data stored in a database entry can include, for example, a patient identifier, medical condition, a treatment type, a treatment status, questions from the questionnaire and corresponding answers provided by patients in response, and a patient score.

In some implementations, the application server 120 can compute a score for a patient data such as a received medical questionnaire form, by processing the question-answer pairs, answers, or both, provided by the user. In some implementations, the application server 120 additionally relies on information collected from a representative population, whose members have characteristics similar to the user (for example, age, gender, race, ethnicity, among others) to compute the score. For example, for a medical questionnaire form, the application server 120 computes a score based on answers provided by the patient, and comparing the patient's profile to a population of individuals with similar profile characteristics. In such implementations, the computed score provides an assessment of the patient's health condition, in comparison to others with similar characteristics. The score can be a value determined by the application server 120, or other computer, based on an analysis of a completed, or partially completed, patient questionnaire. The score can be used to classify a patient that is associated with a particular patient questionnaire into a particular class of patients.

In some implementations, the computed score is sent as the score and analysis report 154 to the user device 110, which presents the score as well as other possible analysis on the GUI 112. The score and analysis report 154 can include data that describes an analysis of the score, for example, to facilitate better understanding of the score by the user 150, such as patient's relative health condition compared to others with similar characteristics or health condition tracked over a given time. In the example of FIG. 1, the analysis can also include recommendations for the user 150. Such recommendations can include, for example, treatment options for health conditions.

In some implementations, other forms of interactive elements are used in place of the answer options 116 on the scale shown in FIG. 1. For example, selectable text bubbles, radio buttons, hover-activated icons or the like may be used. The present specification is not limited to the particular use of answer options 116 as shown in FIG. 1. Any relevant form of interactive element may be used as part of an animated questionnaire to extract data from a user such as the user 150.

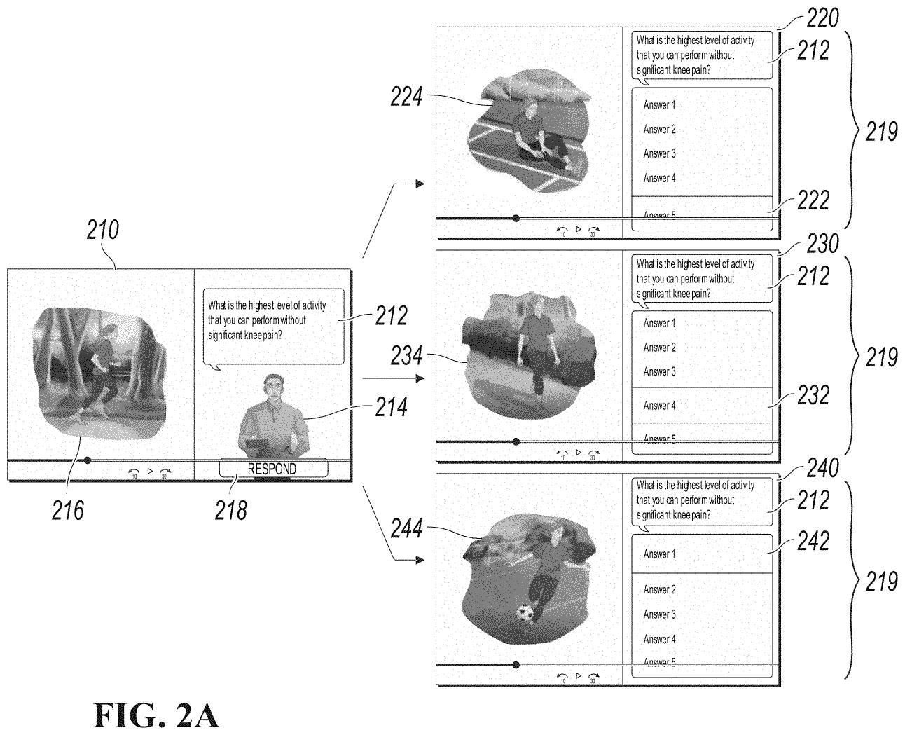

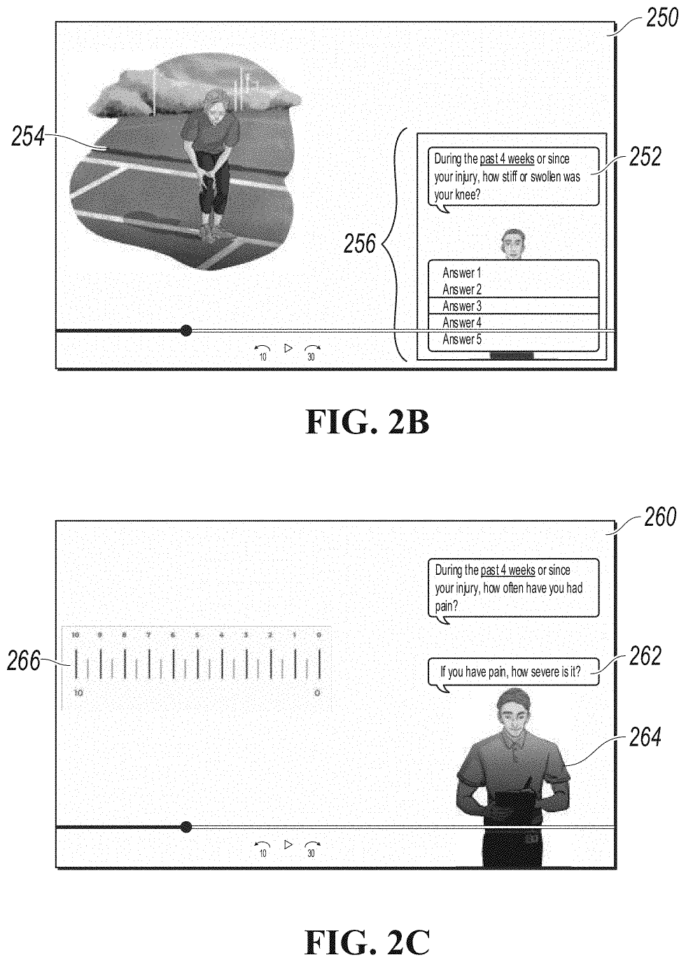

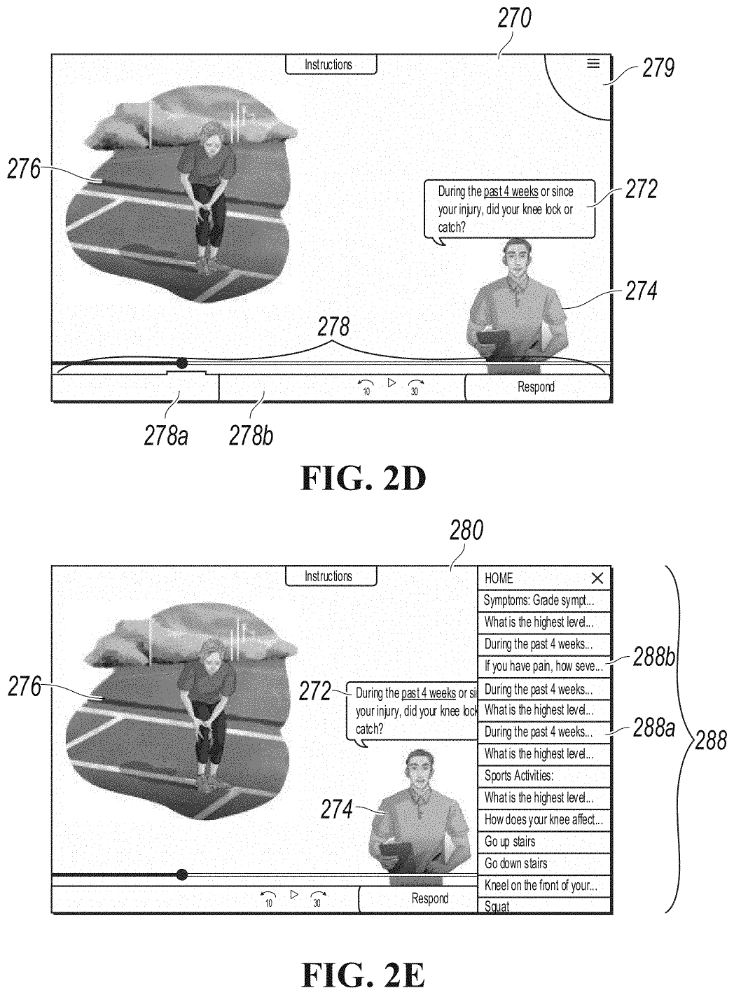

FIGS. 2A-2E illustrate examples of panels 210-280 of a questionnaire form with graphical visualizations of questions and corresponding answer options that are provided for display on a graphical user interface shown on a user device. In some implementations, the panels 210-280 are for a questionnaire form that is shown in the GUI 112 of the user device 110. Accordingly, the panels 210-280 are described below as being provided by the system 100. However, in other implementations, the panels 210-280 correspond to questionnaire forms shown on other user devices in other systems.

As described above, in some implementations, the system 100 is used to collect data using digital medical questionnaire forms. The panels 210-280 provide an example of an implementation of a medical questionnaire form, showing questions and answer options for a medical condition. However, in other implementations, the system 100 is used to collect data for other purposes, for example, consumer product data collection. In such implementations, panels with different graphical visualizations are provided for corresponding digital questionnaire forms, with relevant question and answer options that are presented using animation styles or interactivity options, or both, as discussed below with respect to the illustrated example of a medical questionnaire form.

A medical questionnaire form can be presented to a patient in a digital version to obtain information about a medical condition experienced by the patient. For example, a patient may be experiencing a knee problem, and the medical questionnaire may ask a series of questions to find out about the patient's condition. Panel 210 illustrates one such question being provided for display on the GUI 112. As shown, in some implementations, the panel 210 includes a graphical visualization 212 of a question that is presented in textual form. In some implementations, the question can be presented in audible form. For example, in some cases, the question can be presented audibly along with a graphical rendering of the question.

In some implementations, a graphical visualization 214 of a character is shown in the panel 210 as reading out the question. The graphical visualization 214 can include animation, for example, movement of the head, arms, hands or lips, or changes in facial expression, if the character in the visualization 214, as the question is presented.

In some implementations, a graphical visualization 216 of a medical condition that is a focus of the question is presented in the panel. For example, when the question is related to a medical condition for the knee, as shown by the illustrated example, the visualization 216 can provide a graphical rendering of running or walking, or some other activity that is associated with the medical condition for the knee. The visualization 216 can provide context for the question being asked, for example, by helping the target user of the questionnaire form, for example, the patient, better understand the question. In some cases, the graphical visualization 216 can include a character performing an activity corresponding to the question. For example, as shown by the illustrated example, the visualization 216 can include a person running or walking, or performing some other activity that is associated with the medical condition for the knee.

The user can select to respond to the question presented on the panel 210 by interacting with an input option provided on the panel, such as input option 218. Although only one input option is shown by the illustrated example, in some implementations, a panel can include more than one input option, for example, options to skip the question or go back to the previous question, among others.

If the user selects the input option 218 to respond to the question presented on the panel 210, in some implementations, different answer options are presented for the user to select from. The user can scroll through the answer options to review each option, for example, by hovering over each option using an input mechanism such as a mouse cursor, a stylus or a finger. As the user scrolls through the answer options, temporarily selecting each answer option to review, panels with different graphical visualizations are provided for display on the GUI 112, with the graphical visualizations in a panel corresponding to the answer option presently being reviewed by the user.

Panels 220-240 illustrate examples of different graphical visualizations provided for display on the GUI 112 corresponding to different answer options reviewed by the user to respond to question 212. In the example of FIG. 2A, the text corresponding to the question 212 reads, “What is the highest level of activity that you can perform without significant knee pain?” In other implementations, other questions or groups of questions may be provided to a user. The example of FIG. 2A serves only to explain a particular example of a particular question and answer set. It is contemplated that any relevant question or questions, and any relevant answers related to the question or questions may be provided depending on implementation.

As shown by the panels 220-240, in some implementations, answer options are presented using a pop-up menu 219 showing the different answer options, for example, answer options 222, 232 and 242. When the user temporarily selects answer option 222 to review, graphical visualization 224 is provided for display using the panel 220. When the user temporarily selects a different answer option to review, for example, answer option 232 or 242, a different graphical visualization 234 or 244, respectively, is provided for display using the panel 230 or 240, respectively. In some implementations, one or more of the panels 220-240 also includes the graphical visualization 212 of the question corresponding to the answer options shown using the menu 219.

The visualizations 224, 234 or 244 provide a graphical rendering of a condition or activity corresponding to the answer option presently selected. As shown by the illustrative example, when the question is for a medical condition related to knee pain, the answer options in the menu 219 may ask the user to select the most strenuous activity the user can perform with the knee pain. The answer option 222 is to indicate that the user cannot perform any physical activity due to the knee pain, and the corresponding graphical visualization 224 depicts a character who is not engaged in any physical activity that can tax the knee. In contrast, the answer option 232 is for performing light activities like walking, housework or yardwork. When the user hovers over or otherwise selects answer option 232, the graphical panel provided for display on the GUI 112 changes to panel 230, such that graphical visualization 234 corresponding to answer option 232 can be displayed, depicting a character engaged in such a light physical activity.

In changing selections, the graphical panel provided for display on the GUI 112 changes to match the currently selected answer. For example, in changing selections from the answer option 222 to the answer option 232, the graphical panel provided for display on the GUI 112 changes from panel 220 to panel 230. Considering each answer option as if it were on a scale of activity level, the answer option 222 would be on a low end of such a scale as it relates to not being able to move or having much pain when moving. Possible text related to the answer option 222 could read, “Unable to perform any of the above activities due to knee pain.” The answer option 232 is less extreme corresponding to slightly more strenuous exercise. Possible text related to the answer option 232 could read, “Light activities like walking, housework or yard work.” The answer option 242 would be on the high end of such a scale as it corresponds to performing strenuous activities like jumping or playing soccer. Possible text related to the answer option 242 can read, for example, “Very strenuous activities like jumping or pivoting as in basketball or soccer.”

In other implementations, different answer options with different corresponding text or other representations, including non-textual based answer options that make use of symbols or other representations to convey meaning, may be provided. Images shown on the graphical panel may correspond with the ideas presented in any possible answer option. When the user scrolls from answer option 222 or 232 to answer option 242, the graphical panel provided for display on the GUI 112 changes to panel 240, such that graphical visualization 244 corresponding to answer option 242 can be displayed, depicting a character engaged in a strenuous physical activity, such as playing soccer.

In some implementations, one or more of the graphical visualizations 216, 224, 234, or 244, or other graphical visualizations described in this disclosure, can include animation, for example, movement of the limbs or changes in facial expression of the included characters, as the corresponding question or answer option is presented. Panel 250 in FIG. 2B illustrates an example of animation that can be provided in a graphical visualization corresponding to different answer options. As shown, the panel 250 presents answer options for question 252. In the example of FIG. 2B, the question is, “During the past 4 weeks or since your injury, how stiff or swollen was your knee?” Pop-up menu 256 presents answer options indicating varying degrees of severity of the knee condition. For example, answer options in one implementation may include one or more of: “Not at all”, “Mildly”, “Moderately”, “Very”, or “Extremely”. When the user scrolls through the different answer options, the facial expression of the character included in the graphical visualization 254 changes commensurate with the severity corresponding to the currently selected answer option. In this case, the question is not related to physical activities that can be performed by the user. As such, the general posture of the character in the graphical visualization remains the same across the various panels that are provided, but each of the answer options is contextualized by animating the facial expression of the character. For example, for increasing severity, the facial expression of the character becomes more strained, indicating more pain.

In some implementations, answer options may be contextualized by other actions of the character. For example, a set of answer options may be directed to a question asking a user to rank knee pain. An answer option indicating severe knee pain may be selected by a user from among the set of answer options. When selected, a graphic panel similar to the panel 250 may show the character grabbing their knee in addition to showing a facial expression of pain. In another panel, associated with a different answer option indicating less severe pain, the character may not be grabbing their knee but may simply show a facial expression indicating a corresponding level of pain relative to other answer options in the set of answer options provided. The facial expression together with the additional actions of the character, in this case grabbing their knee, may help contextualize the answer for a user answering the given question.

In some implementations, different characters are included in the graphical visualization depending on the profile of the target user, such as gender or age, among others. For example, when the user completing the questionnaire is an adult woman or man, the character shown in the visualizations 216, 224, 234 and 244, can be that of an adult woman or a man, respectively. In some cases, the character shown in visualizations, such as visualization 214, can change depending on the given user completing the questionnaire. For example, if the character shown in visualization 214 is a doctor, the character can be customized to appear as a doctor associated with a given user such as a primary care physician of the user, a specialist, or another character known to the user, including fictional characters. When the user completing the questionnaire is a teenager, the character shown in the visualizations 216, 224, 234 and 244, can be that of a teenager. In such implementations, the application processes that are executed to provide the questionnaire form on the GUI 112 obtains information about the target user, such as name, age, gender, among others, before presenting the panels of the questionnaire. In some implementations, the character shown by one or more of the visualizations can be a customized representation of the user, for example, by using a rendering of a photograph of the user.

The answer options can be shown in the panels using formats other than a pop-up menu. In some implementations, the answer options are presented as a series of radio buttons, each of which can be selected by the user, for example, as shown with respect to answer option 116. In some implementations, the answer options are presented as a sliding scale, for example, answer option 262 as shown in the panel 260 of FIG. 2C. Other suitable formats are also possible. In each of these formats, panels with different graphical visualizations are provided for display on the GUI as the user scrolls or browses through the different answer options, in a manner similar to that discussed above.

In some implementations, when the user selects an answer option as her response to a question, animation is used to indicate the system recording the user's response. For example, as shown by FIG. 2C, a question 262 can ask about the severity of the user's pain (e.g., “If you have pain, how severe is it?”), and the panel 260 includes graphical visualization 264 of a character as reading out the question 262. The user can select a number on the scale 266 to indicate her perceived severity of pain in response to the question 262. Following the user's response, the character in the graphical visualization 264 can be animated to indicate recording of the user's answer. For example, the character can be shown to write down the user's answer using a pen and paper on a clipboard. Another example may show the character nodding by movement of the head or otherwise indicating the response has been recorded.

In some implementations, after the user's answer to a question has been recorded, application processes that generate the questionnaire form determine if there are additional questions left to be answered. In case there are unanswered questions, the application processes provide panels for to an unanswered question and corresponding answer options for display on the GUI 112, for example, in a manner similar to that described above with respect to panels 210-250.

Accordingly, in the manner described above, a questionnaire form can be provided for display on a GUI of a user device in conversational form, for example, depicting an interaction with a character asking a question. Questions and answer options are presented with graphical visualizations of corresponding activities, so that the user can visualize the context for the questions and answer options. In some implementations, animation is provided in the graphical visualizations, for example, depicting physical activities described in the questions and answer options, or depicting variations in facial expression or posture depending on the intensity of the activity in the question, to provide better context for the rationale for the questions and answer options.

As described above, in some implementations, the questionnaire form provided for display on the GUI 112 of device 110 is a digital version of a standard questionnaire form, which is conventionally presented as text on a physical sheet of paper, for example, a sheet of paper with a medical questionnaire form provided to a patient upon check in at a medical facility, such as a physician's office. In such implementations, the digital questionnaire form provided for display on the GUI 112 is presented in a manner that maintains the integrity and validation of the standard questionnaire, while obtaining information from the user in a conversational format. For example, the text for questions or answer options, or both, shown using the panels on the GUI 112 can be the same as the text for corresponding questions and answer options in a standard questionnaire form. In some implementations, an order in which the questions or answer options, or both, are provided for display on the GUI 112 using the panels is the same as an order in which questions or answer options are presented in a standard questionnaire form.

In some implementations, additional input options are provided in the panels with the questions and answer options. The additional input options are configured to enable the user to navigate through the questionnaire form efficiently, for example, in a non-sequential manner. FIGS. 2D and 2E illustrate examples of panels 270 and 280, respectively, with the additional input options. In a manner similar to that described with respect to panel 210, the panel 270 includes a graphical visualization 272 of a question, with a graphical visualization 274 of a character shown reading out the question, and a graphical visualization 276 of a medical condition that is a focus of the question is presented in the panel. Additionally, the panel 270 presents a horizontal navigation bar 278, which includes a plurality of tiles, for example, tiles 278a and 278b.

The panel 280 in FIG. 2E also includes the graphical visualization 272 of the question, along with the graphical visualization 274 of the character shown reading out the question and the graphical visualization 276 of the medical condition that is the focus of the question is presented in the panel. However, in contrast to panel 270, the panel 280 also presents a vertical navigation bar 288, which includes a plurality of tiles, for example, tiles 288a and 288b. In some implementations, the vertical navigation bar 288 is displayed based on user selection of the input option 279, shown in the panel 270.

The navigation bar 278 of FIG. 2D or navigation bar 288 of FIG. 2E may be used by the user to navigate to specific questions or panels within a questionnaire. For example, the user may select tile 278a to answer a first question and select tile 278b to answer a second question. In some cases, a user may only be required to or wish to answer a subset of questions. The navigation bar can thus help a user find and answer relevant questions. In some cases, the navigation bar includes information on one or more of the tiles 278a and 278b indicating the associated content. For example, tile 278a can include information associated with the first question so that the user, based on seeing the information associated with the first question, knows to click on the tile 278a to access a corresponding panel associated with the tile 278a or answer the first question associated with the tile 278a. For example, the tile 278a can include a topic of question, a question identifier, a question itself, or other non-textual visualization (e.g., cartoon indicating a body part or a set of questions).

Each tile in the navigation bar 278, or the navigation bar 288, corresponds to a question in the questionnaire form. In some implementations, the tile corresponding to the question currently shown in the panel is highlighted using a suitable mechanism. For example, the tile 278a in horizontal navigation bar 278 can correspond to the question 272. Accordingly, in the panel 270, the tile 278a is highlighted, for example, by being raised in profile compared to other tiles, or having a different color or animation, or some other suitable mechanism. As another example, the tile 288a in vertical navigation bar 288 can correspond to the question 272. Accordingly, in the panel 280, the tile 288a is highlighted, for example, by being presented in a different color compared to other tiles in the navigation bar 288.

The user can navigate the questionnaire form using the navigation bar 278 or the navigation bar 288, or both. For example, using the navigation bar 278, the user can jump from the question panel corresponding to tile 278a to a different panel that corresponds to tile 278b, which is separated from the tile 278a by at least one other tile in a sequential order in which questions are presented in the form. Similarly, using the navigation bar 288, the user can jump from the question panel corresponding to tile 288a to a different panel that corresponds to tile 288b, which is separated from the tile 288a by at least one other tile in the sequential order in which questions are presented in the form.

In some implementations, a characteristic of each tile in the navigation bar 278, or the navigation bar 288, or both, can be used to indicate the answer selected by the user in response to the question associated with the tile. For example, the color, saturation or hue of a tile can be changed. For example, a tile can be a deep red in color if the selected answer is “severe pain.” In some implementations, text corresponding to the selected answer is overlaid on the representation of the tile, for example, as shown with respect to tiles 288a and 288b.

In some implementations, when the user completes the questionnaire form and the answer options selected by the user are recorded, the application processes that are executed to provide the digital questionnaire form computes a score for the user based on the selected answer options such as the score and analysis report 154 of FIG. 1. Characteristics of the user, for example, age, gender, marital status, among others, can be taken into account in computing the score. Different weights may be assigned to different answer options, or different characteristics, or both, to compute the score. In some implementations, a panel is provided with graphical visualizations to present the score to the user. The graphical visualizations can include a character, for example, representing a physician, who reads the score to the user. In some implementations, an analysis of the score is also presented, which can include a comparison to a population of other users with similar profile characteristics.

In some implementations, the digital representation of the questionnaire form includes gamification content to enhance engagement of the user. In this context, gamification refers to providing the panels of the questionnaire form for display on the GUI using a format of an online or video game. The game format can include a storyline simulating a real-world environment, for example, a typical workday in the life of an individual in the city, or a day in a summer camp. One or more characters can be included in the storyline to represent the user. In some cases, a user can customize the appearance of a character included in the storyline. A character included in the storyline may have a similar appearance to a given user such as a user currently engaged with the GUI display on a device such as the user device 110. As the user interacts with the game, questions and corresponding answer options are presented to the user, for example, as described above with respect to panels 210-280. The user can progress through the storyline by selecting answer options for the questions. Additional characters can be included in the storyline as progress is made, to maintain interest of the user. The user can earn rewards for playing the game, for example, in the form of improved scores, points that can be reimbursed for merchandise, digital stickers, among others. Using gamification content for the digital questionnaire forms can be useful to engage users with limited attention spans, for example, to engage young children who are patients in a pediatric facility.

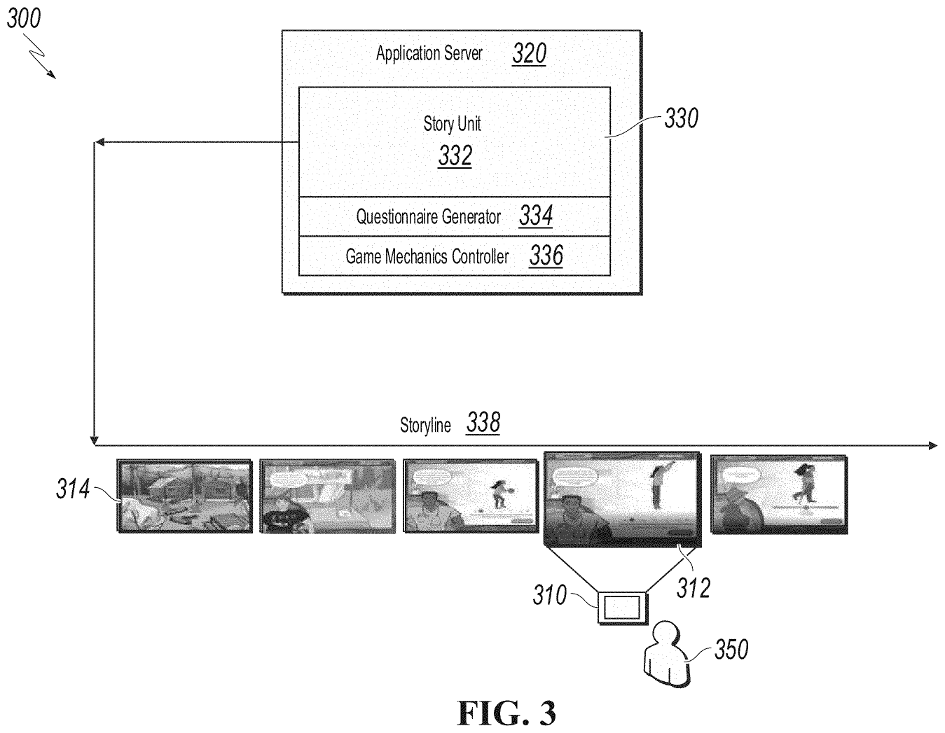

FIG. 3 is a diagram of an example of a system 300 for conversational data collection using a digital questionnaire form with gamification. The system 300 includes a user device 310 that is communicably connected to a remote network server 320. The network server 320 executes one or more application processes 330 to generate a digital questionnaire form, including providing, for display on a GUI of the user device 310, panels, for example, panels 312 and 314, with graphical visualizations representing questions and answer options for the questionnaire form. A user 350 interacts with the panels through the GUI of the device 310 to respond to the questionnaire form.

In some implementations, the system 300 is similar to the system 100. In such implementations, the user device 310 is similar to the user device 110, the application server 320 is similar to the application server 120, and the user 350 is similar to the user 150. In the following sections, although the one or more application processes 330 are described as being executed by the application server 320, in other implementations, the one or more application processes 330 are executed locally on the user device 310.

In some implementations, the one or more application processes 330 include a story unit 332, a questionnaire generator 334 and a game mechanics controller 336. The story unit 332 stores one or more storylines that can be provided along with questions and answer options for a questionnaire form. The conversational prompts generator is configured to provide the questions and answer options, which are embedded in the storyline that is presented. The game mechanics controller 336 is configured to gamify the presentation of the questions and answer options in the storyline, for example, by dynamically changing the sequence in which questions are presented based on prior answers from the user, keeping scores for the user, or providing rewards, among others.

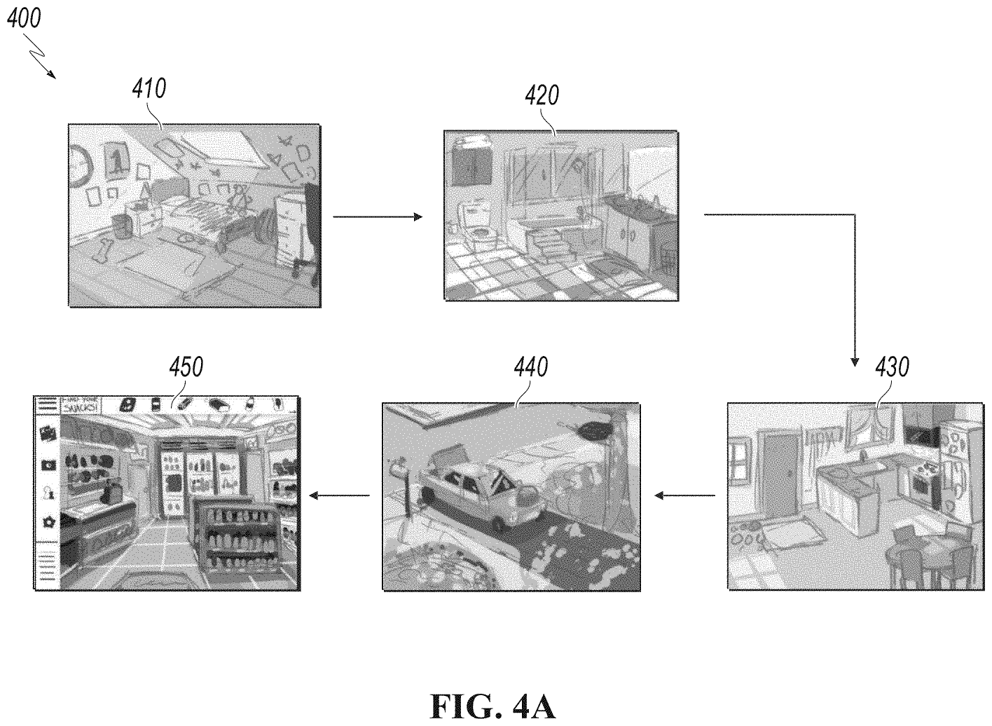

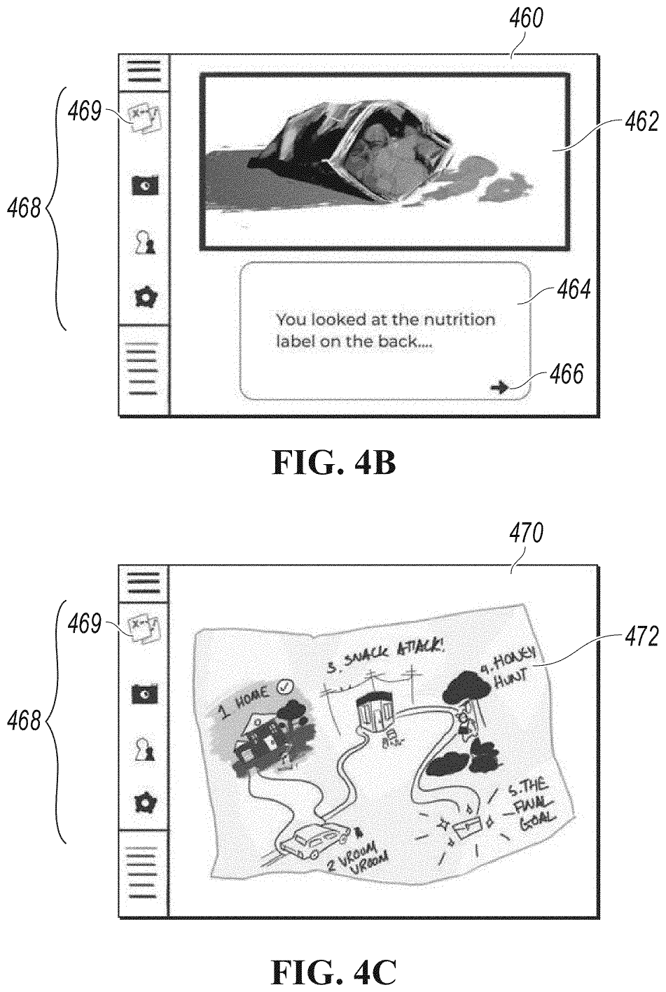

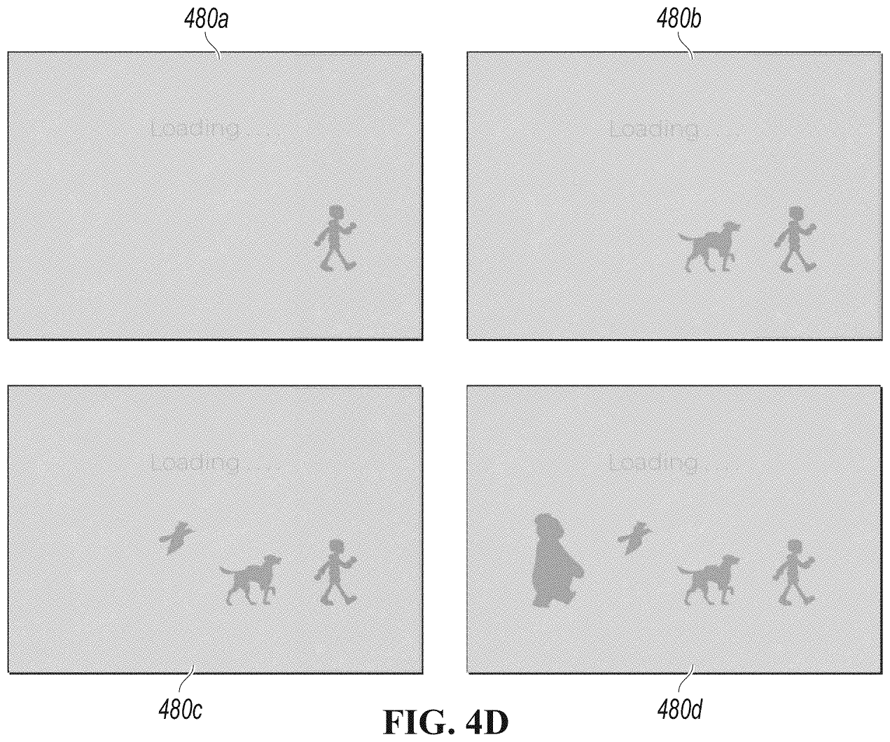

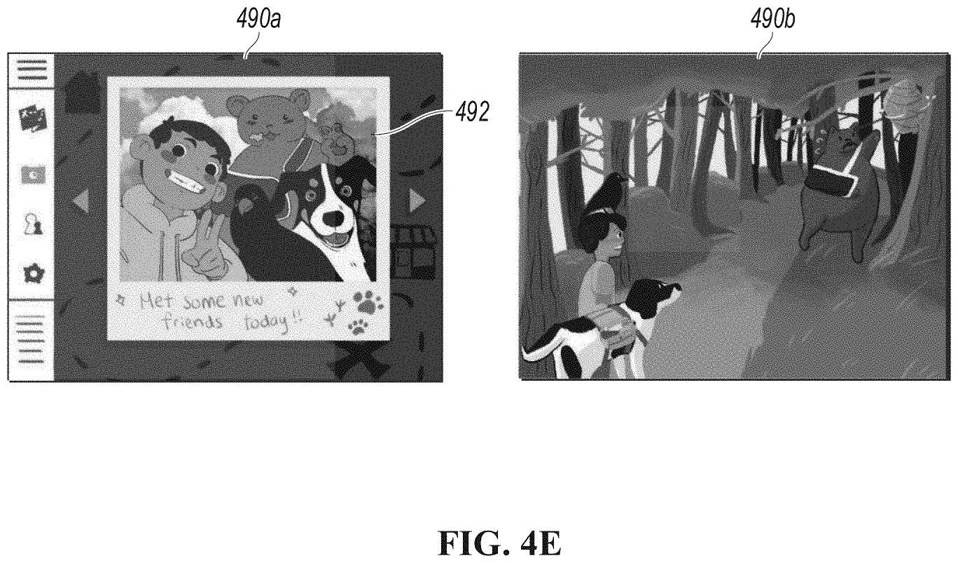

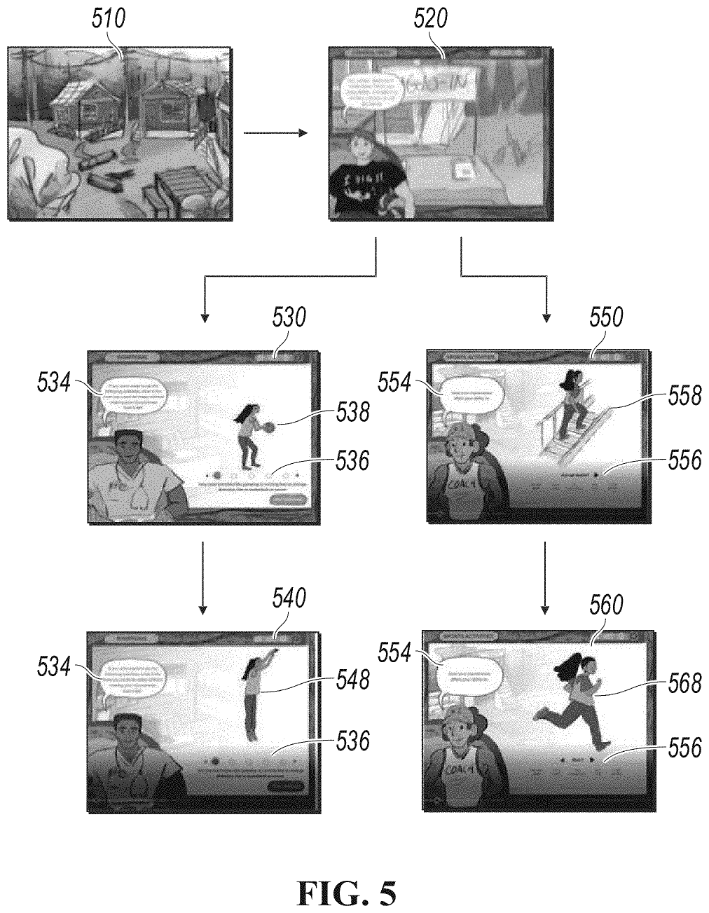

The application server 320 provides panels for a storyline 338 from the story unit 332, to the user device 310. The storyline 338 includes a number of panels, for example panels 312 and 314, which collectively form a cohesive narrative of a story used to engage the user 350 with questions and answer options for the questionnaire form. The storyline 338 is one of the plurality of storylines that are stored by the story unit. In some implementations, the one or more application processes 330 can dynamically select a particular storyline 338 based on a profile of the user 350. For example, when the user completing the questionnaire form is a young child, the storyline 338 can be a day at summer camp. On the other hand, when the user completing the questionnaire form is an adult, the storyline 338 can follow a timeline of a workday. Examples of storylines with questions and answer options are described with respect to FIGS. 4A-4F and FIG. 5. In some implementations, available storylines are presented to the user 350, who selects the particular storyline 338 from the available options.

The panels 312 and 314 are displayed on the GUI of the user device 310, each panel including a question or one or more answer options, or both, in a manner similar to that described above with respect to panels 210-280. The user 350 views the panels on the GUI of the user device 310 and interacts with the game by providing user inputs for responding to questions or selecting answer options. In some implementations, the display sequence of the panels is controlled by the user inputs. In other implementations, the display sequence of the panels follows a predictable order, for example, the same order in which questions and answer options are presented in a corresponding standard questionnaire form.

In some implementations, the panels enable the user 350 to control the panels using additional input options, for example, horizontal or vertical navigation bars, or both, as described with respect to panels 270 and 280. By using the navigation bars, the user 350 can move through the storyline 338 from one panel to another panel at a user's own pace, pause to ponder, observe the visualization shown in the panel, or return to a previous panel, as is possible with pages of a book.

In some implementations, the panels are used to present the questions and corresponding answer options as interactive challenges to the player, and the storyline waits for input from the player in response to the challenges. In some implementations, the game mechanics controller 336 game-related activities when the user 350 responds to a question or selects an answer option. For example, the game mechanics controller 336 can compute a score based on the response from the user 350, and provide the score for display on the GUI of the user device 310. Additionally or alternatively, the game mechanics controller 336 can generate rewards for the user, and provide information about the rewards for display on the GUI, along with the panels.

As one example of a storyline used to gamify a questionnaire form, FIGS. 4A-4E illustrate examples of panels 410-490B that are provided to display, on a graphical user interface shown on a user device, a digital questionnaire form in the form of a storyline with gamification. In some implementations, the panels 410-490B are for a questionnaire form that is presented on the GUI of the user device 310. Accordingly, the panels 410-490B are described below as being provided by the system 300. However, in other implementations, the panels 410-490B correspond to questionnaire forms shown on other user devices in other systems.

The panels 410-450 shown in FIG. 4A illustrate an example of a storyline simulating a real-world environment that is used to present questions and answer options for a questionnaire form. For example, as shown, the storyline can depict a character getting ready for work or school in the morning, with panels 410, 420, 430, 440 and 450 provided in a sequential order to show, respectively, the user waking up in the morning; brushing teeth and getting ready in the bathroom; having breakfast in the kitchen; getting ready to drive to work or school; and stopping by a convenience store to buy snacks. In some implementations, the story unit 332 generates the graphical visualizations of the simulated environment shown by the panels 410-450.

In some implementations, the questionnaire generator 334 embeds questions and answer options for a questionnaire form within the panels of the storyline depicted with respect to the panels 410-450. For example, when the panel 450 is displayed on the GUI, the user may provide an input indicating purchase of snacks from the convenience store visualized by the panel 450. Upon receiving the input from the user, the questionnaire generator 334 can coordinate with the story unit 332 to provide the panel 460 of FIG. 4B for display on the GUI of the user device. As shown, the panel 460 includes a graphical visualization 462 of a snack that the user indicated as having been purchased, along with a corresponding question 464. The user can select the input option 466 to respond to the question, at which time a new panel with answer options can be provided for display.

In some implementations, the panel 460 includes additional input options 468 for the user to navigate the storyline. For example, the input option 469 can correspond to an overview of the storyline for the game. When the user provides an input indicating selection of the input option 469, the questionnaire generator 334 can coordinate with the story unit 332 to provide the panel 470 of FIG. 4C for display on the GUI of the user device. As shown, the panel 470 includes a graphical visualization 472 that provides an overview of the storyline used for presenting the questionnaire form.

As described above, the user progresses through the game aspect of the questionnaire form by selecting answer options as responses to questions presented using the panels. The game mechanics controller 336 manages the gamification aspect of the questionnaire, including, for example, generating rewards for the user. In some implementations, the rewards include introduction of new characters in the storyline as the user completes answers to certain questions of the questionnaire form. FIG. 4D illustrates examples of panels 480A-480D that are provided for display to indicate rewards earned by the user at various stages in the presentation of the storyline, upon successful completion of certain questions of the questionnaire form. In some cases, the user completing the gamified questionnaire form can be a young boy. In these cases, as indicated by panel 480A, initially, the storyline can include a single character representing the user. Subsequently, upon answering certain questions, the user can be rewarded by introduction of a second character, for example, a dog as a pet, which is indicated by providing panel 480B for display. At further stages of the storyline, the user can be rewarded by introduction of additional characters, for example, a bird and a bear, which are respectively indicated by providing panels 480C and 480D for display at the corresponding stages.

The characters can be integrated into the storyline following their inclusion at different stages. FIG. 4E illustrates examples of panels 490A and 490B with graphical visualizations that incorporate the new characters into the storyline. Panel 490A includes graphical visualization 492 showing the character representing the user, along with characters representing the dog, the bird, and the bear. Panel 490B includes a graphical visualization of an activity in the storyline being performed by these characters.

As another example of a storyline used to gamify a questionnaire form, FIG. 5 illustrates examples of panels 510-560 that are provided to display, on a graphical user interface shown on a user device, a digital questionnaire form in the form of a storyline with gamification. In some implementations, the panels 510-560 are for a questionnaire form that is presented on the GUI of the user device 310. Accordingly, the panels 510-560 are described below as being provided by the system 300. However, in other implementations, the panels 510-560 correspond to questionnaire forms shown on other user devices in other systems.

The panels 510-560 illustrate an example of a storyline simulating a real-world environment that is used to present questions and answer options for a questionnaire form. For example, as shown, the storyline can depict a character going to summer camp, with panels 510 and 520 provided as introductory panels in a sequential order to describe the storyline or questionnaire, or both, to the user and obtain profile information from the user. In some implementations, the story unit 332 generates the graphical visualizations of the simulated environment shown by the panels 510-520.

In some implementations, depending on answers provided by the user to a question, the narrative of the storyline can change. For example, the game mechanics controller 336 can process the information provided by the user in response to a question presented using the panel 520, and control the storyline to follow a sequence of either panels 530 and 540, or a sequence of panels 550 and 560, based on the information provided by the user.

In any of these sequences, the questionnaire generator 334 embeds questions and answer options for the questionnaire form within the panels 530, 540, 550 and 560. For example, the panel 530 provides a graphical visualization 534 of a question being asked by a physician character, and a graphical visualization of a scale 536 with answer options to the question. The user can browse the different answer options in the scale 536. Depending on the answer option reviewed by the user, graphical visualizations of corresponding activities are also presented by the panels, for example, graphical visualizations 538 and 548 presented on panels 530 and 540, respectively, representing activities corresponding to different answer options provided by the scale 536. As shown, the question represented by the graphical visualization 534 is related to an orthopedic medical condition, e.g., asking for information about activities the user can perform with her injured knee. The various answer options shown with respect to the scale 536 indicates different physical activities that require different levels of knee strength. Accordingly, the answer option selected by the user represents the user's response about the physical activity she can comfortably perform with her injured knee, thereby providing information about the extent and current status of her knee injury.

Similarly, panels 550 and 560 present a graphical visualization 554 of a question being asked by another character, and a graphical visualization 556 with answer options to the question. Depending on the answer option reviewed by the user, graphical visualizations 558 and 568 are presented on panels 550 and 560, respectively, representing activities corresponding to different answer options. The characters and the activities in the visualizations fit in with the simulated environment of the storyline, for example, showing a camp counselor in panel 520 or a camp sports coach in panels 550 and 560.

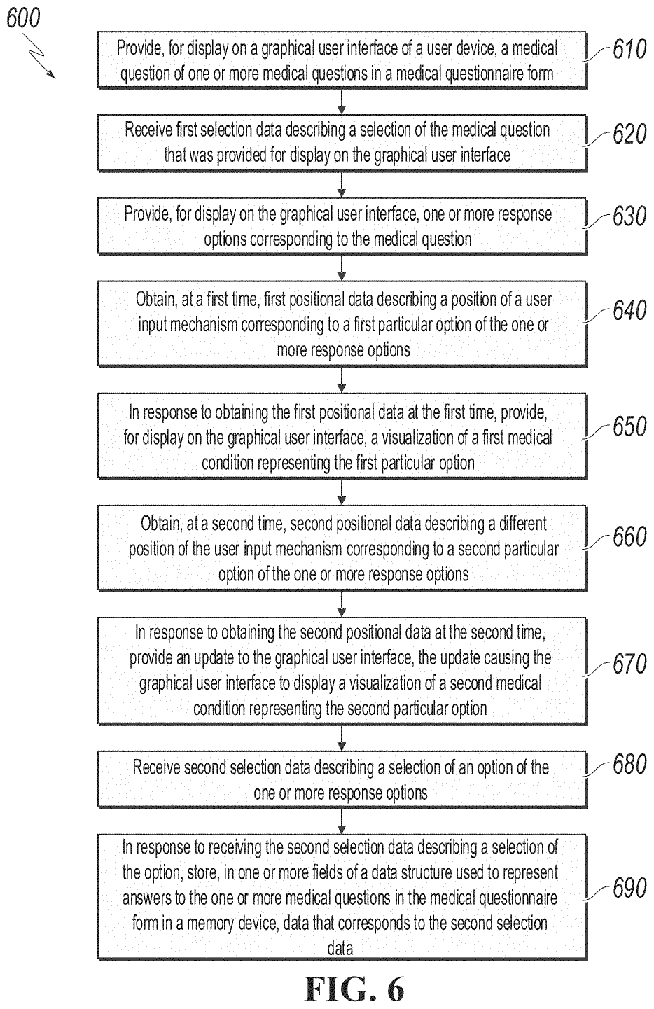

FIG. 6 illustrates an example of a process 600 for conversational data collection using a digital questionnaire form with graphical visualizations. In some implementations, the process 600 is performed by the user device 110 or the application server 120, or both, to collect patient data for a medical questionnaire form using panels with graphical visualizations corresponding to the questions and answer options. Accordingly, the following sections describe the process 600 as being performed by components of the system 100. However, the process 600 also may be performed by other systems.

The process 600 starts when a medical question of one or more medical questions in a medical questionnaire form is provided for display on a graphical user interface of a user device (610). For example, a panel 210 is displayed on GUI 112 of user device 110 with a graphical visualization 212 of a medical question, along with a graphical visualization of 214 of a character asking the question and graphical visualization 216 of a medical condition that is the focus of the question 212.

First selection data describing a selection of the medical question that was provided for display on the graphical user interface is received (620). For example, user selection of input option 218 presented on the panel is received, indicating user intent to respond to the question 212.

One or more response options corresponding to the medical question are provided for display on the graphical user interface (630). For example, in response to the user selection of input option 218 to respond to the question 212 presented on the panel 210, a pop-up menu 219 of answer options are presented using one of the panels 220-240.

At a first time, first positional data describing a position of a user input mechanism corresponding to a first particular option of the one or more response options is obtained (640). For example, the user can scroll through the answer options presented using the pop-up menu 219. At a first time, the user input temporarily selects answer option 222, for example, by hovering the user input mechanism over the answer option 222 or otherwise selecting the answer option 222.

In response to the obtaining the first positional data at the first time, a visualization of a first medical condition representing the first particular option is provided for display on the graphical user interface (650). When the user temporarily selects answer option 222 to review, graphical visualization 224 is provided for display using the panel 220, with graphical visualization 224 providing a graphical rendering of a medical condition or activity corresponding to answer option 222.

At a second time, second positional data describing a different position of the user input mechanism corresponding to a second particular option of the one or more response options is obtained (660). For example, scrolling through the answer options presented using the pop-up menu 219, the user provides input at a second time temporarily selecting answer option 232.

In response to obtaining the second positional data at the second time, an update to the graphical user interface is provided, where the update causes the graphical user interface to display a visualization of a second medical condition representing the second particular option (670). For example, when the user scrolls from answer option 222 to answer option 232, the graphical panel provided for display on the GUI 112 changes from panel 220 to panel 230, such that graphical visualization 234 corresponding to answer option 232 is displayed, depicting a character engaged in such a physical activity corresponding to answer option 232.

Second selection data describing a selection of an option of the one or more response options is received (680). For example, the user provides input indicating selection of one of the answer options shown using pop-up menu 219, as the user's response to question 212.

In response to receiving the second selection data describing a selection of the option, data that corresponds to the second selection data is stored in one or more fields of a data structure used to represent answers to the one or more medical questions in the medical questionnaire form in a memory device (690). For example, the answer provided by the user in response to question 212 is stored in memory of the user device 110. Additionally or alternatively, the answers selected by the user for the questions in the questionnaire form are collected from the memory of the user device 110 and sent, over the network 140, to the application server 120 as document 152. The application server 120 stores the completed questionnaire form that has been answered by a user as an entry, e.g., a database record, in the database 130. The answer options selected by the user are stored as data in one or more fields corresponding to the entry.

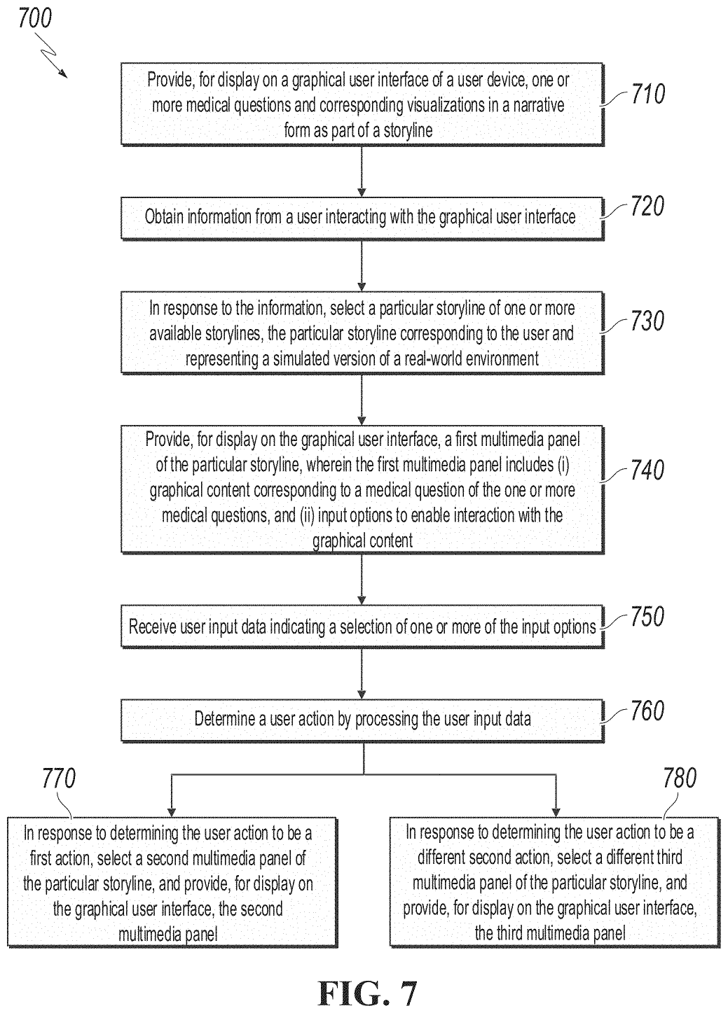

FIG. 7 illustrates an example of a process 700 for conversational data collection using a digital questionnaire form with gamification. In some implementations, the process 700 is performed by the user device 310 or the application server 320, or both, to collect patient data for a medical questionnaire form by presenting the questions and answer options in a storyline with gamified content. Accordingly, the following sections describe the process 700 as being performed by components of the system 300. However, the process 700 also may be performed by other systems.

The process 700 starts when one or more medical questions and corresponding visualizations are provided, for display on a graphical user interface of a user device, in a narrative form as part of a storyline (710). For example, panel 520 is provided for display on the GUI of the user device 310, with the panel presenting a question for a medical questionnaire form within a storyline simulating a real-world environment, e.g., a summer camp.

Information from a user interacting with the graphical user interface is obtained (720). For example, the game mechanics controller 336 processes the information provided by the user in response to a question presented using the panel 520.

In response to the information, a particular storyline of one or more available storylines is selected, the particular storyline corresponding to the user and representing a simulated version of a real-world environment (730). For example, the game mechanics controller 336 processes the information provided by the user in response to a question presented using the panel 520, and controls the storyline to follow a sequence of either panels 530 and 540, or a sequence of panels 550 and 560, based on the information provided by the user. Additionally or alternatively, the story unit 332 selects a particular storyline from one of available storylines, e.g., a workday or summer camp, depending on information obtained from the user, such as the user's age.

A first multimedia panel of the particular storyline is provided for display on the graphical user interface, where the first multimedia panel includes (i) graphical content corresponding to a medical question of the one or more medical questions, and (ii) input options to enable interaction with the graphical content (740). For example, the game mechanics controller 336 controls the storyline to follow the sequence starting with panel 530, and provides panel 530 for display on the GUI of the user device 310. The questionnaire generator 334 embeds a question and answer options for the questionnaire form within the panel 530, providing a graphical visualization 534 of a question being asked by a physician character, and a graphical visualization of a scale 536 with answer options to the question. The user is enabled to browse the different answer options in the scale 536; depending on the answer option being reviewed by the user, graphical visualizations of corresponding activities are also presented, e.g., graphical visualization 538, representing a physical activity corresponding to different answer options provided by the scale 536.

A user input data indicating a selection of one or more of the input options is received (750). For example, a user input is received for panel 530 or 540 indicating user selection of one of the answer options provided by the scale 536, as the user's response to the question 534.

A user action is determined by processing the user input data (760). For example, as described with respect to panels 530 and 540, the answer option selected by the user from the scale 536 represents the user's response about a physical activity she can comfortably perform with her injured knee.

A second multimedia panel of the particular storyline is selected in response to determining the user action to be a first action, and the second multimedia panel is provided for display on the graphical user interface (770). For example, the questionnaire generator 334 determines the status of the user's knee injury based on the answer option selected by the user from the scale 536. In some cases, upon determining that the injury is mostly healed, the questionnaire generator 334 selects a new panel that depicts more strenuous physical activities, e.g., playing soccer as shown with respect to panel 240, for display on the GUI. In selecting the new panel, the questionnaire generator 334 attempts to extract, with greater precision, the extent of strenuous physical activities the user can do, given that her knee injury is determined to be mostly healed.

On the other hand, in response to determining the user action to be a different second action, a different third multimedia panel of the particular storyline is selected and provided for display on the graphical user interface (780). For example, the questionnaire generator 334 determines the status of the user's knee injury based on the answer option selected by the user from the scale 536. In some cases, upon determining that the injury is still painful, the questionnaire generator 334 selects a new panel that depicts more gentle physical activities, e.g., walking or housework as shown with respect to panel 230, for display on the GUI.

In this manner, depending on the user selection of an answer option to a question, one of several available sequences of further questions is selected, such that the storyline can follow different narratives for different users. In some implementations, the game mechanics controller 336 also computes and updates a score for the user for every question that is answered. In some implementations, the game mechanics controller 336 provides rewards to the user at various stages of answers to questions in the questionnaire form, as the user progresses with the questionnaire form through the storyline.

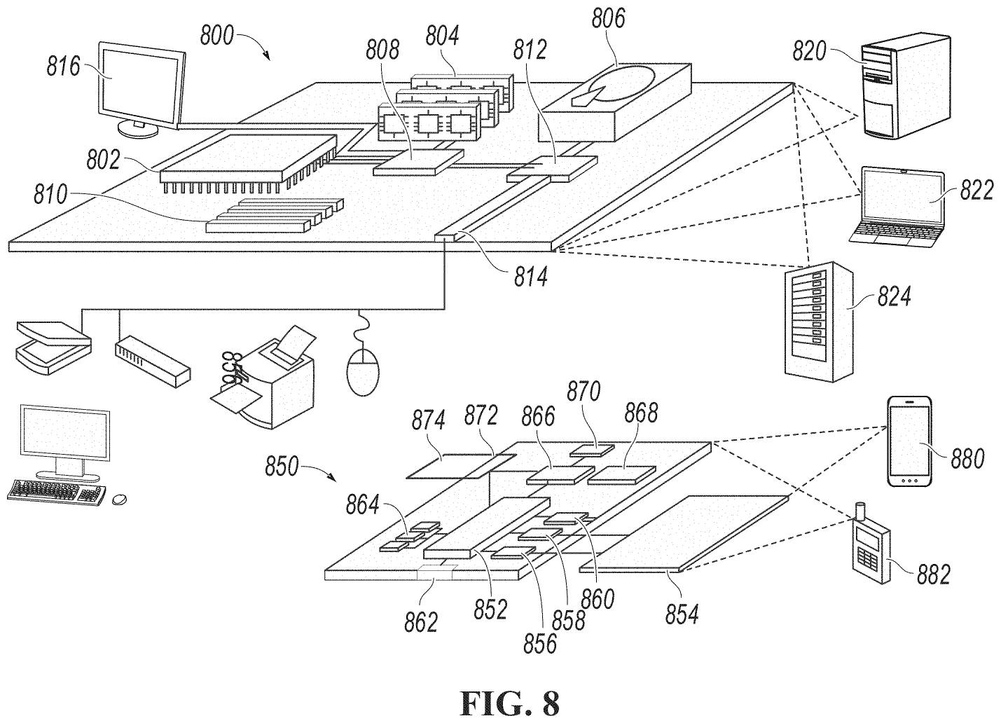

FIG. 8 is a diagram of computing devices 800 and 850 that can be used to implement a system for conversational data collection using digital questionnaire forms. Computing device 800 is intended to represent various forms of digital computers, such as laptops, desktops, workstations, personal digital assistants, servers, blade servers, mainframes, and other appropriate computers. Computing device 850 is intended to represent various forms of mobile devices, such as personal digital assistants, cellular telephones, smartphones, and other similar computing devices. Additionally, computing device 800 or 850 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 800 includes a processor 802, memory 804, a storage device 806, a high-speed interface 808 connecting to memory 804 and high-speed expansion ports 810, and a low speed interface 812 connecting to low speed bus 814 and storage device 806. Each of the components 802, 804, 806, 808, 810, and 812, are interconnected using various busses, and can be mounted on a common motherboard or in other manners as appropriate. The processor 802 can process instructions for execution within the computing device 800, including instructions stored in the memory 804 or on the storage device 808 to display graphical information for a GUI on an external input/output device, such as display 816 coupled to high speed interface 808. In other implementations, multiple processors and/or multiple buses can be used, as appropriate, along with multiple memories and types of memory. Also, multiple computing devices 800 can be connected, with each device providing portions of the necessary operations, for example, as a server bank, a group of blade servers, or a multi-processor system.

The memory 804 stores information within the computing device 800. In one implementation, the memory 804 is a volatile memory unit or units. In another implementation, the memory 804 is a non-volatile memory unit or units. The memory 804 can also be another form of computer-readable medium, such as a magnetic or optical disk.

The storage device 808 is capable of providing mass storage for the computing device 800. In one implementation, the storage device 808 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 804, the storage device 808, or memory on processor 802.

The high speed controller 808 manages bandwidth-intensive operations for the computing device 800, while the low speed controller 812 manages lower bandwidth intensive operations. Such allocation of functions is exemplary only. In one implementation, the high-speed controller 808 is coupled to memory 804, display 816, for example, through a graphics processor or accelerator, and to high-speed expansion ports 810, which can accept various expansion cards (not shown). In the implementation, low-speed controller 812 is coupled to storage device 808 and low-speed expansion port 814. The low-speed expansion port, which can include various communication ports, for example, USB, Bluetooth, Ethernet, wireless Ethernet can be coupled to one or more input/output devices, such as a keyboard, a pointing device, microphone/speaker pair, a scanner, or a networking device such as a switch or router, for example, through a network adapter. The computing device 800 can be implemented in a number of different forms, as shown in the figure. For example, it can be implemented as a standard server 820, or multiple times in a group of such servers. It can also be implemented as part of a rack server system 824. In addition, it can be implemented in a personal computer such as a laptop computer 822. Alternatively, components from computing device 800 can be combined with other components in a mobile device (not shown), such as device 850. Each of such devices can contain one or more of computing device 800, 850, and an entire system can be made up of multiple computing devices 800, 850 communicating with each other.

The computing device 800 can be implemented in a number of different forms, as shown in the figure. For example, it can be implemented as a standard server 820, or multiple times in a group of such servers. It can also be implemented as part of a rack server system 824. In addition, it can be implemented in a personal computer such as a laptop computer 822. Alternatively, components from computing device 800 can be combined with other components in a mobile device (not shown), such as device 850. Each of such devices can contain one or more of computing device 800, 850, and an entire system can be made up of multiple computing devices 800, 850 communicating with each other.

Computing device 850 includes a processor 852, memory 864, and an input/output device such as a display 854, a communication interface 866, and a transceiver 868, among other components. The device 850 can also be provided with a storage device, such as a micro-drive or other device, to provide additional storage. Each of the components 850, 852, 864, 854, 866, and 868, are interconnected using various buses, and several of the components can be mounted on a common motherboard or in other manners as appropriate.

The processor 852 can execute instructions within the computing device 850, including instructions stored in the memory 864. The processor can be implemented as a chipset of chips that include separate and multiple analog and digital processors. Additionally, the processor can be implemented using any of a number of architectures. For example, the processor 810 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 850, such as control of user interfaces, applications run by device 850, and wireless communication by device 850.

Processor 852 can communicate with a user through control interface 858 and display interface 856 coupled to a display 854. The display 854 can be, for example, a TFT (Thin-Film-Transistor Liquid Crystal Display) display or an OLED (Organic Light Emitting Diode) display, or other appropriate display technology. The display interface 856 can comprise appropriate circuitry for driving the display 854 to present graphical and other information to a user. The control interface 858 can receive commands from a user and convert them for submission to the processor 852. In addition, an external interface 862 can be provide in communication with processor 852, so as to enable near area communication of device 850 with other devices. External interface 862 can provide, for example, for wired communication in some implementations, or for wireless communication in other implementations, and multiple interfaces can also be used.

The memory 864 stores information within the computing device 850. The memory 864 can be implemented as one or more of a computer-readable medium or media, a volatile memory unit or units, or a non-volatile memory unit or units. Expansion memory 874 can also be provided and connected to device 850 through expansion interface 872, which can include, for example, a SIMM (Single In Line Memory Module) card interface. Such expansion memory 874 can provide extra storage space for device 850, or can also store applications or other information for device 850. Specifically, expansion memory 874 can include instructions to carry out or supplement the processes described above, and can include secure information also. Thus, for example, expansion memory 874 can be provide as a security module for device 850, and can be programmed with instructions that permit secure use of device 850. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 864, expansion memory 874, or memory on processor 852 that can be received, for example, over transceiver 868 or external interface 862.

Device 850 can communicate wirelessly through communication interface 866, which can include digital signal processing circuitry where necessary. Communication interface 866 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 868. In addition, short-range communication can occur, such as using a Bluetooth, Wi-Fi, or other such transceiver (not shown). In addition, GPS (Global Positioning System) receiver module 870 can provide additional navigation- and location-related wireless data to device 850, which can be used as appropriate by applications running on device 850.

Device 850 can also communicate audibly using audio codec 860, which can receive spoken information from a user and convert it to usable digital information. Audio codec 860 can likewise generate audible sound for a user, such as through a speaker, for example, in a handset of device 850. Such sound can include sound from voice telephone calls, can include recorded sound, for example, voice messages, music files, etc. and can also include sound generated by applications operating on device 850.

The computing device 850 can be implemented in a number of different forms, as shown in the figure. For example, it can be implemented as a cellular telephone 880. It can also be implemented as part of a smartphone 882, personal digital assistant, or other similar mobile device.

Embodiments of the subject matter, the functional operations and the processes described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively, or in addition, the program instructions can be encoded on an artificially generated propagated signal, for example, a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” encompasses all kinds of apparatus, devices, and machines for processing data, including by way of example a programmable processor, a computer, or multiple processors or computers. The apparatus can include special purpose logic circuitry, for example, an FPGA (field programmable gate array) or an ASIC (application specific integrated circuit). The apparatus can also include, in addition to hardware, code that creates an execution environment for the computer program in question, for example, code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (for example, one or more scripts stored in a markup language document), in a single file dedicated to the program in question, or in multiple coordinated files (for example, files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, for example, an FPGA (field programmable gate array) or an ASIC (application specific integrated circuit).

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, for example, magnetic, magneto optical disks, or optical disks. However, a computer need not have such devices. Moreover, a computer can be embedded in another device, for example, a mobile telephone, a personal digital assistant (PDA), a mobile audio or video player, a game console, a Global Positioning System (GPS) receiver, or a portable storage device (for example, a universal serial bus (USB) flash drive), to name just a few.

Computer readable media suitable for storing computer program instructions and data include all forms of nonvolatile memory, media and memory devices, including by way of example semiconductor memory devices, for example, EPROM, EEPROM, and flash memory devices; magnetic disks, for example, internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, for example, a CRT (cathode ray tube) or LCD (liquid crystal display) monitor, for displaying information to the user and a keyboard and a pointing device, for example, a mouse or a trackball, by which the user can provide input to the computer. Other kinds of devices can be used to provide for interaction with a user as well; for example, feedback provided to the user can be any form of sensory feedback, for example, visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, for example, as a data server, or that includes a middleware component, for example, an application server, or that includes a front end component, for example, a client computer having a graphical user interface or a Web browser through which a user can interact with an implementation of the subject matter described in this specification, or any combination of one or more such back end, middleware, or front end components. The components of the system can be interconnected by any form or medium of digital data communication, for example, a communication network. Examples of communication networks include a local area network (“LAN”) and a wide area network (“WAN”), for example, the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

1. A method comprising:
providing, for display on a graphical user interface of a user device, a medical question of one or more medical questions in a medical questionnaire form;
receiving first selection data describing a selection of the medical question that was provided for display on the graphical user interface;
in response to receiving the first selection data describing the selection of the medical question, providing, for display on the graphical user interface, one or more response options corresponding to the medical question;
obtaining, at a first time, first positional data describing a position of a user input mechanism corresponding to a first particular option of the one or more response options;
in response to the obtaining the first positional data at the first time, providing, for display on the graphical user interface, a visualization of a first medical condition representing the first particular option;
obtaining, at a second time, second positional data describing a different position of the user input mechanism corresponding to a second particular option of the one or more response options;
in response to obtaining the second positional data at the second time, providing an update to the graphical user interface, wherein the update causes the graphical user interface to display a visualization of a second medical condition representing the second particular option;
receiving second selection data describing a selection of an option of the one or more response options; and
in response to receiving the second selection data describing the selection of the option, storing, in one or more fields of a data structure used to represent answers to the one or more medical questions in the medical questionnaire form in a memory device, data that corresponds to the second selection data.

2. The method of claim 1, further comprising:
providing for display, on the graphical user interface, each of the one or more medical questions in a same order as an order in which the one or more medical questions are included in the medical questionnaire form.

3. The method of claim 2, wherein the medical questionnaire form is a digital representation of a standard medical form, and wherein the order of the one or more medical questions in the medical questionnaire form corresponds to an order of presentation of medical questions in the standard medical form.

4. The method of claim 1, wherein the one or more response options correspond to different physical activities, the first particular option corresponding to a first physical activity and the second particular option corresponding to a different second physical activity, and wherein
providing the visualization of the first medical condition comprises providing, for display on the graphical user interface, a visualization of the first physical activity, and
providing the visualization of the second medical condition comprises providing, for display on the graphical user interface, a visualization of the second physical activity.

5. The method of claim 1, wherein the one or more response options correspond to different intensity levels of a physical condition experienced by a user, the first particular option corresponding to a first intensity level and the second particular option corresponding to a different second intensity level, and wherein
providing the visualization of the first medical condition comprises providing, for display on the graphical user interface, a visualization of the first intensity level, and
providing the visualization of the second medical condition comprises providing, for display on the graphical user interface, a visualization of the second intensity level.

6. The method of claim 1, wherein providing, for display on the graphical user interface, at least one of: the medical question, the first medical condition, or the second medical condition, comprises providing, for display on the graphical user interface, a multimedia animation.

7. The method of claim 1, further comprising providing, for display on the graphical user interface, the one or more medical questions and the visualizations in a narrative form as part of a storyline, the method further comprising:
obtaining information from a user interacting with the graphical user interface;
in response to the information, selecting a particular storyline of one or more available storylines, the particular storyline corresponding to the user and representing a simulated version of a real-world environment;
providing, for display on the graphical user interface, a first multimedia panel of the particular storyline, wherein the first multimedia panel includes (i) graphical content corresponding to a medical question of the one or more medical questions, and (ii) input options to enable interaction with the graphical content;
receiving user input data indicating a selection one of the input options;
determining a user action by processing the user input data;
in response to determining the user action to be a first action:
selecting a second multimedia panel of the particular storyline, and
providing, for display on the graphical user interface, the second multimedia panel; and
in response to determining the user action to be a different second action:
selecting a different third multimedia panel of the particular storyline, and
providing, for display on the graphical user interface, the third multimedia panel.

8. The method of claim 1, further comprising:
determining whether at least one medical question of the one or more medical questions in the medical questionnaire form is unanswered; and
based on determining that at least one medical question in the medical questionnaire form is unanswered, providing, for display on the graphical user interface, an unanswered medical question.

9. The method of claim 1, further comprising:
determining whether at least one medical question of the one or more medical questions in the medical questionnaire form is unanswered; and
based on determining that no medical question is unanswered:
reviewing selection data corresponding to the one or more medical questions stored in the one or more fields of the data structure;
computing a score using the selection data; and
providing, for display on the graphical user interface, a summary of the medical questionnaire form, the summary including the computed score and a corresponding medical analysis.

10. A non-transitory computer-readable medium storing software comprising instructions executable by one or more computers which, upon such execution, cause the one or more computers to perform operations comprising:
providing, for display on a graphical user interface of a user device, a medical question of one or more medical questions in a medical questionnaire form;
receiving first selection data describing a selection of the medical question that was provided for display on the graphical user interface;
in response to receiving the first selection data describing the selection of the medical question, providing, for display on the graphical user interface, one or more response options corresponding to the medical question;
obtaining, at a first time, first positional data describing a position of a user input mechanism corresponding to a first particular option of the one or more response options;
in response to the obtaining the first positional data at the first time, providing, for display on the graphical user interface, a visualization of a first medical condition representing the first particular option;
obtaining, at a second time, second positional data describing a different position of the user input mechanism corresponding to a second particular option of the one or more response options;
in response to obtaining the second positional data at the second time, providing an update to the graphical user interface, wherein the update causes the graphical user interface to display a visualization of a second medical condition representing the second particular option;
receiving second selection data describing a selection of an option of the one or more response options; and
in response to receiving the second selection data describing the selection of the option, storing, in one or more fields of a data structure used to represent answers to the one or more medical questions in the medical questionnaire form in a memory device, data that corresponds to the second selection data.

11. The non-transitory computer-readable medium of claim 10, wherein the operations further comprise:
providing for display, on the graphical user interface, each of the one or more medical questions in a same order as an order in which the one or more medical questions are included in the medical questionnaire form.

12. The non-transitory computer-readable medium of claim 10, wherein the one or more response options correspond to different physical activities, the first particular option corresponding to a first physical activity and the second particular option corresponding to a different second physical activity, and wherein
providing the visualization of the first medical condition comprises providing, for di splay on the graphical user interface, a visualization of the first physical activity, and
providing the visualization of the second medical condition comprises providing, for display on the graphical user interface, a visualization of the second physical activity.

13. The non-transitory computer-readable medium of claim 10, wherein the one or more response options correspond to different intensity levels of a physical condition experienced by a user, the first particular option corresponding to a first intensity level and the second particular option corresponding to a different second intensity level, and wherein
providing the visualization of the first medical condition comprises providing, for display on the graphical user interface, a visualization of the first intensity level, and
providing the visualization of the second medical condition comprises providing, for display on the graphical user interface, a visualization of the second intensity level.

14. The non-transitory computer-readable medium of claim 10, wherein the operations comprise providing, for display on the graphical user interface, the one or more medical questions and the visualizations in a narrative form as part of a storyline, and wherein the operations further comprise:
obtaining information from a user interacting with the graphical user interface;
in response to the information, selecting a particular storyline of one or more available storylines, the particular storyline corresponding to the user and representing a simulated version of a real-world environment;
providing, for display on the graphical user interface, a first multimedia panel of the particular storyline, wherein the first multimedia panel includes (i) graphical content corresponding to a medical question of the one or more medical questions, and (ii) input options to enable interaction with the graphical content;
receiving user input data indicating a selection one of the input options;
determining a user action by processing the user input data;
in response to determining the user action to be a first action:
selecting a second multimedia panel of the particular storyline, and
providing, for display on the graphical user interface, the second multimedia panel; and
in response to determining the user action to be a different second action:
selecting a different third multimedia panel of the particular storyline, and
providing, for display on the graphical user interface, the third multimedia panel.

15. A data processing system for conversational data collection, the data processing system comprising:
one or more processors; and
one or more computer storage devices storing instructions that, when executed by the one or more processors, cause the one or more processors to perform operations comprising:
providing, for display on a graphical user interface of a user device, a medical question of one or more medical questions in a medical questionnaire form;
receiving first selection data describing a selection of the medical question that was provided for display on the graphical user interface;
in response to receiving the first selection data describing the selection of the medical question, providing, for display on the graphical user interface, one or more response options corresponding to the medical question;
obtaining, at a first time, first positional data describing a position of a user input mechanism corresponding to a first particular option of the one or more response options;
in response to the obtaining the first positional data at the first time, providing, for display on the graphical user interface, a visualization of a first medical condition representing the first particular option;
obtaining, at a second time, second positional data describing a different position of the user input mechanism corresponding to a second particular option of the one or more response options;
in response to obtaining the second positional data at the second time, providing an update to the graphical user interface, wherein the update causes the graphical user interface to display a visualization of a second medical condition representing the second particular option;
receiving second selection data describing a selection of an option of the one or more response options; and
in response to receiving the second selection data describing the selection of the option, storing, in one or more fields of a data structure used to represent answers to the one or more medical questions in the medical questionnaire form in a memory device, data that corresponds to the second selection data.

16. The data processing system of claim 15, wherein the one or more response options correspond to different physical activities, the first particular option corresponding to a first physical activity and the second particular option corresponding to a different second physical activity, and wherein
providing the visualization of the first medical condition comprises providing, for display on the graphical user interface, a visualization of the first physical activity, and
providing the visualization of the second medical condition comprises providing, for display on the graphical user interface, a visualization of the second physical activity.

17. The data processing system of claim 15, wherein the one or more response options correspond to different intensity levels of a physical condition experienced by a user, the first particular option corresponding to a first intensity level and the second particular option corresponding to a different second intensity level, and wherein
providing the visualization of the first medical condition comprises providing, for display on the graphical user interface, a visualization of the first intensity level, and
providing the visualization of the second medical condition comprises providing, for display on the graphical user interface, a visualization of the second intensity level.

18. The data processing system of claim 15, wherein the operations comprise providing, for display on the graphical user interface, the one or more medical questions and the visualizations in a narrative form as part of a storyline, and wherein the operations further comprise:
obtaining information from a user interacting with the graphical user interface;
in response to the information, selecting a particular storyline of one or more available storylines, the particular storyline corresponding to the user and representing a simulated version of a real-world environment;
providing, for display on the graphical user interface, a first multimedia panel of the particular storyline, wherein the first multimedia panel includes (i) graphical content corresponding to a medical question of the one or more medical questions, and (ii) input options to enable interaction with the graphical content;
receiving user input data indicating a selection one of the input options;
determining a user action by processing the user input data;
in response to determining the user action to be a first action:
selecting a second multimedia panel of the particular storyline, and
providing, for display on the graphical user interface, the second multimedia panel; and
in response to determining the user action to be a different second action:
selecting a different third multimedia panel of the particular storyline, and
providing, for display on the graphical user interface, the third multimedia panel.

19. The data processing system of claim 15, wherein the operations further comprise:
determining whether at least one medical question of the one or more medical questions in the medical questionnaire form is unanswered; and
based on determining that at least one medical question in the medical questionnaire form is unanswered, providing, for display on the graphical user interface, an unanswered medical question.

20. The data processing system of claim 15, wherein the operations further comprise:
determining whether at least one medical question of the one or more medical questions in the medical questionnaire form is unanswered; and
based on determining that no medical question is unanswered:
reviewing selection data corresponding to the one or more medical questions stored in the one or more fields of the data structure;
computing a score using the selection data; and
providing, for display on the graphical user interface, a summary of the medical questionnaire form, the summary including the computed score and a corresponding medical analysis.
